(19) United States
(12) Patent Application Publication
(10) Pub. No.: US 2021/0346686 A1
(43) Pub. Date: Nov. 11, 2021

(54) Implantable Electrode Lead for a Curved Implantation Path

(71) Applicant: BIOTRONIK SE & Co. KG, Berlin (DE)

(72) Inventors: Michael FRIEDRICH, Kleinmachnow (DE); Gernot KOLBERG, Berlin (DE); Ingo WEISS, Berlin (DE)

(73) Assignee: BIOTRONIK SE & Co. KG, Berlin (DE)

(21) Appl. No.: 17/286,266

(22) PCT Filed: Oct. 18, 2019
(86) PCT No.: PCT/EP2019/078342

§ 371 Date: Apr. 16, 2021

(30) Foreign Application Priority Data
Oct. 19, 2018 (DE) 10 2018 126 037.7

(51) Int. Cl. A61N 1/05; A61N 1/375

(52) U.S. Cl. CPC A61N 1/057; A61N 2001/058; A61N 1/37518; A61N 1/0563

(57) ABSTRACT

An implantable electrode lead having proximal and distal ends, comprising an electrode lead body and a pulling element. The pulling element has first and second ends. At the first end, the pulling element is connected to the distal end of the electrode lead or in the vicinity of the electrode lead distal end; referred to as the distal region of the electrode lead. Proceeding from the joining site between the first end of the pulling element and the electrode lead, the pulling element extends from the first end thereof toward the second end thereof to the proximal end of the electrode lead. The pulling element, proceeding from the joining site, extends outside the electrode lead body. A tensile force exerted onto the second end of the pulling element exerts a bending moment onto the electrode lead distal region. This bending moment results in bending of the electrode lead distal region.

### CROSS-REFERENCE TO RELATED APPLICATIONS

This application is the United States national phase under 35 U.S.C. § 371 of PCT International Patent Application No. PCT/EP2019/078342, filed on Oct. 18, 2019, which claims the benefit of German Patent Application No. 10 2018 126 037.7, filed on Oct. 19, 2018, the disclosures of which are hereby incorporated by reference herein in their entireties.

### TECHNICAL FIELD

The present invention relates to an implantable extravascular electrode lead that can be connected to a pulse generator and steered on a defined path during implantation.

### BACKGROUND

The vast majority of defibrillators implanted today use implantable electrode leads, which extend via the vascular system into the heart of the patient so as to stop critical cardiological conditions, such as tachycardia, there by the deliberate delivery of high-energy pulses (shocks) to the myocardium. An alternative concept for an implantable defibrillator, in contrast, provides placing the electrode lead inside the body, but outside the vascular system. The electrode leads are usually arranged subcutaneously or submuscularly in the extrathoracic region.

So as to enable optimal placement of the extravascular implantable defibrillator and of the electrode leads thereof, it is indispensable for the electrode leads to extend along curved or bent paths. The presently known electrode leads for subcutaneous or submuscular implantation are suitable for being pushed forward subcutaneously or submuscularly only in a rectilinear manner in a given step. If the electrode lead is to extend along a defined path having a bend, an incision is required at the site of the bend so as to push the portion of the electrode lead beyond the bend forward in a rectilinear manner. In the case of multiple bends or curves, it is thus necessary to make multiple incisions. The electrode lead is consecutively tunneled through multiple incisions in different directions, thus being given the correct position. Multiple incisions, however, increase the risk of infection and are cosmetically disadvantageous.

Another known option is for an electrode lead to be pretensioned internally, as is known, for example, in the case of vascular J electrodes, which brings the electrode lead into a curved state. However, the designs for precurved electrode leads are limited. The pretensioned electrode lead will only assume the predefined shape on the scale that the anatomy allows. This makes later curving difficult to define. Moreover, such a pretensioned electrode lead cannot be steered with respect to the curve since the curve is fixed with this design.

Another known option is to place the electrode lead into the desired curved or bent position using precurved or steerable introducer sheaths. Even though the use of steerable sheaths is possible for this purpose, these are challenging and cumbersome in terms of the handling thereof. Additionally, sheaths, and in particular steerable sheaths, are expensive.

Finally, the option exists to guide the electrode lead using a curved stylet. However, similarly to the pretensioned electrode leads described above, the design here is not only limited, but also heavily dependent on the particular anatomic circumstances, and thus difficult to define.

The present disclosure is directed toward overcoming one or more of the above-mentioned problems, though not necessarily limited to embodiments that do.

### SUMMARY

It is thus an object to create an electrode lead in which the position of the implantable electrode lead is steerable in the tissue onto a defined path, without necessitating additional incisions.

At least this object is achieved by an implantable electrode lead according to claim 1.

The implantable extravascular electrode lead comprises an electrode lead body and an elongated pulling element. The electrode lead body extends from a distal end of the electrode lead to a proximal end of the electrode lead. The pulling element has a first end and a second end, wherein the pulling element, with the first end thereof, is connected to the electrode lead body by means of a joining site at the distal end of the electrode lead body of the electrode lead or in the distal region of the electrode lead, and the pulling element furthermore extends from the joining site to the proximal end of the electrode lead in the elongated state of the electrode lead. The pulling element extends outside the electrode lead body in the process, at least in the distal region of the electrode lead. A tensile force exerted onto the second end of the pulling element exerts a bending moment onto the distal region of the electrode lead, which results in bending of the distal region of the electrode lead.

In another embodiment, the electrode lead body of the electrode lead comprises a guide element for guiding the pulling element on the electrode lead body or in the electrode lead body. In another embodiment, the guide element is a ring, an eyelet or a sleeve.

In another embodiment, the guide element is a channel inside the electrode lead body, which extends at least along a portion of the electrode lead body.

In another embodiment, the distance between the guide element and the joining site between the pulling element and the electrode lead along the electrode lead body is at least 30 mm and no more than 800 mm.

In another embodiment, an electrode pole is arranged on the electrode lead body between the guide element and the joining site. In another embodiment, the electrode pole is designed in the form of a coil, and in particular in the form of a shock coil.

In another embodiment, the electrode lead comprises further electrode poles for stimulating body tissue and for sensing electrical signals of the tissue. These additional electrode poles are preferably implemented as rings.

In another embodiment, the electrode lead comprises a connection device for electrically and mechanically connecting the electrode lead to an implantable pulse generator or defibrillator. The connection device may be an IS4/DF4 plug, for example.

In another embodiment, the electrode lead comprises an engagement device on the electrode lead body thereof. The engagement device is preferably arranged on the electrode lead body in the vicinity of the guide element, or this engagement device forms a part of the guide element.

In another embodiment, a corresponding mating piece for the engagement device is attached at the distal end of the electrode lead or at the joining site between the pulling element and the electrode lead. The mating piece for the engagement device is designed to engage in the engagement device during insertion, so as to establish a mechanical connection between the mating piece for the engagement device and the engagement device. Preferably, in addition to a mechanical connection, an electrical connection can also be established by the connection between the engagement device and the mating piece for the engagement device.

By means of the guide element and the engagement device, a loop or a U shape can be formed by the distal region of the electrode lead.

In another embodiment, the electrode lead comprises a first electrical conductor, which extends along the electrode lead body and connects the at least one electrode pole to the plug in an electrically conducting manner. If the electrode pole is designed as a shock coil, the first conductor can be connected to the proximal end of the electrode pole. Furthermore, this first conductor can be connected to the engagement device in a conducting manner. As an alternative, a second electrical conductor can be arranged in the electrode lead body, which connects the engagement device to the plug in an electrically conducting manner. In addition, a third electrical conductor can be provided in the electrode lead body, which connects the distal end of the electrode pole to the mating piece for the engagement device in an electrically conducting manner. In this way, it is possible to contact the electrode pole, which is preferably implemented as a shock electrode, from both sides. The proximal end of the electrode pole is thus connected via the first conductor, and the distal end of the electrode pole is connected via the second and third conductors, and the engagement device and the mating piece for the engagement device are connected to the plug in an electrically conducting manner. In this way, the conduction losses during the delivery of the shock can be minimized.

In another embodiment, the electrode lead comprises multiple guide elements for the pulling element along the electrode lead body thereof. The guide elements are arranged on the electrode lead body in such a way that the electrode lead body forms a meander when the pulling element is pulled.

In another embodiment, the pulling element can be designed in the form of a thread, a cable, a wire, a bar or a rod.

In another embodiment, the pulling element is resorbable. A (bio)resorbable pulling element shall be understood to mean a pulling element having components that can be decomposed in the body. The bioresorbability has the effect that the pulling element dissolves over a certain time period. In this way, the electrode lead can be explanted more easily.

In another embodiment, the wire or the rod can be precurved.

In another embodiment, the plug comprises a locking unit for locking the pulling element. In this way, the pulling element can be locked at the proximal end of the electrode lead. As a result of this locking unit, it is not possible to pull the pulling element back in the distal direction of the electrode lead. The locking device for the pulling element is preferably reversible.

In another embodiment, the electrode lead can comprise more than one distal end, and thus two separate distal regions. In this case, two pulling elements are provided, which are each guided separately from one another. In this way, the distal regions can be curved individually for each of the two regions.

Another embodiment relates to a system comprising an aforementioned electrode lead and a catheter. The electrode lead is guided in a catheter. The catheter can be implemented in the form of an introducer sheath. The introducer sheath can be steerable in the distal region thereof. Steerable shall be understood to mean that the distal end of the introducer sheath can be bent in at least one direction. The distal end of the introducer sheath can preferably be bent in two spatial directions. In this way, the distal end of the introducer sheath can reach all points on a spherical segment. The introducer sheath is preferably dimensioned with respect to the inside diameter thereof in such a way that the pulling element, together with the electrode lead, can be received by the sheath.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further features, advantages and embodiments of the present invention shall be described hereafter with reference to the figures.

In the drawings:

FIGS. 1A and 1B show an electrode lead comprising an externally guided pulling cable;

FIG. 2 shows an electrode lead comprising an engagement device;

FIG. 3A to 3E show an electrode lead comprising multiple guide elements, which can form a meander;

FIGS. 4A and 4B show an electrode lead comprising two separate distal regions;

FIGS. 5A and 5B show an electrode lead comprising an introducer sheath; and

FIG. 6 shows an introducer sheath having a special design distally.

### DETAILED DESCRIPTION

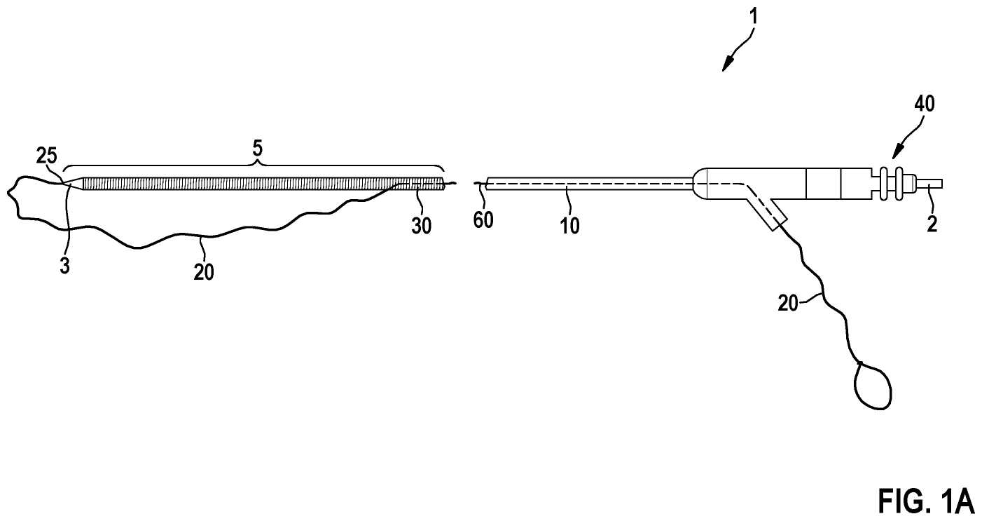

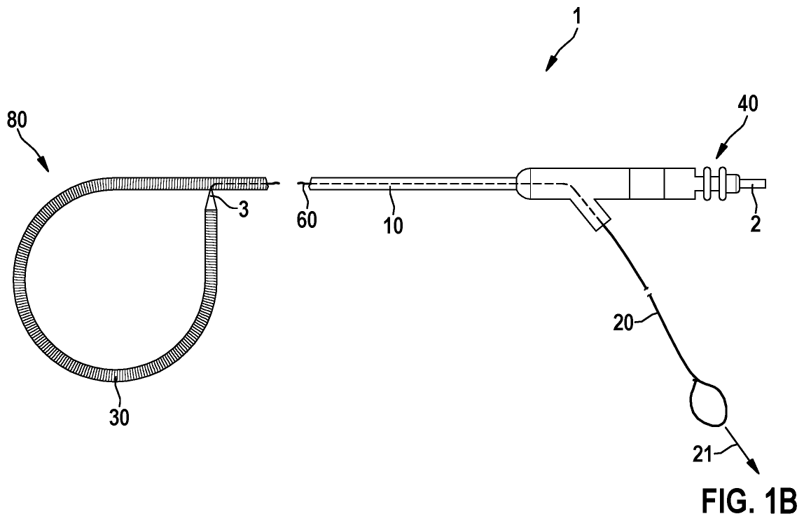

FIG. 1A shows an electrode lead 1 having a proximal end 2 and a distal end 3, wherein a plug 40 for connecting the electrode lead to an active implantable device (not shown) is provided at the proximal end 2. An electrode pole in the form of a coil 30 is arranged in the distal region 5 of the illustrated electrode lead 1. In this case, the coil 30 is provided in the form of a shock coil or in the form of a large-area electrode pole serving as a base electrode or ground. The electrode lead 1 further comprises a pulling element 20, which is attached on the joining site 25 at the distal end 3 of the electrode lead 1. In this exemplary embodiment, the pulling element 20 is designed as a thread or cable. Proceeding from the distal end 3 of the electrode lead 1, the pulling element 20 is received by the guide element 60. In this embodiment, the guide element 60 is provided in the form of a channel inside the electrode lead body 10.

In FIG. 1B, a tensile force 21 is exerted onto the pulling element 20 of the electrode lead 1 in the direction of the arrow. The distal region 5 of the electrode lead 1 is bent into a loop 80 by this tensile force 21. The circular shape of the loop 80 is achieved by the guide element 60. Without the distal guide element 60, it would not be possible without further aids to bring the distal end 3 of the electrode lead 1 to the electrode lead body 10. Moreover, the guide element 60 makes it possible that the exact direction of the tensile force 21 exerted onto the pulling element 20 does not matter for the mechanism to function. Rather, only the portion of the pulling element 20 that is pulled out of the guide element 60 at the proximal end 2 of the electrode lead 1 as a result of the force application 21 must be increased.

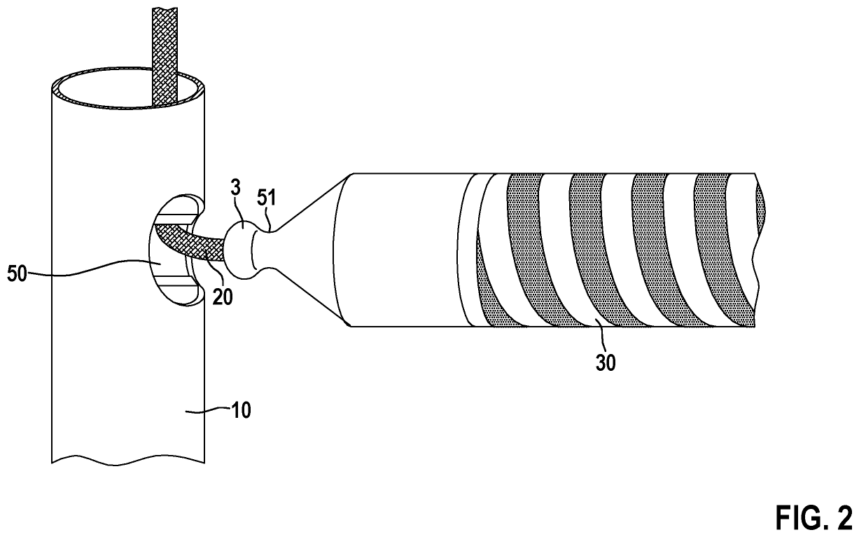

FIG. 2 shows the section of the electrode lead body 10 of the electrode lead 1 on which the pulling element 20 enters the guide element 60. An engagement device 50, in which the mating piece for the engagement device 51 arranged at the distal end 3 of the electrode lead 1 can engage, is present at the entry site into the guide element 60. In this way, the distal end 3 of the electrode lead 1 is mechanically connected to the electrode lead body 10 after being brought close by the pulling element 20. Moreover, the engagement device 50 and the mating piece for the engagement device 51 can be designed in such a way that, in addition to the mechanical connection, also an electrical contact is established between the distal end 3 of the electrode lead and the engagement device 60. In this way, for example, the electrode coil 30 can be electrically connected in two points so as to reduce the voltage drop along the coil 30.

In particular with subcutaneous defibrillators, it is essential that the voltage causing the shock forms a stimulation vector that preferably runs through the heart of the patient. Since it is difficult to optimally place the housing electrode given the large volume of the housing, as an alternative another electrode lead can be used as a counter pole to the stimulation or shock electrode pole. As large an effective electrode surface as possible is advantageous for such electrode leads. The electrode lead 1 shown in FIG. 1B offers an option for increasing the effective electrode surface of an electrode lead 1 by the formation of a loop 80. Another option for increasing the effective electrode surface is implemented by the formation of a meander.

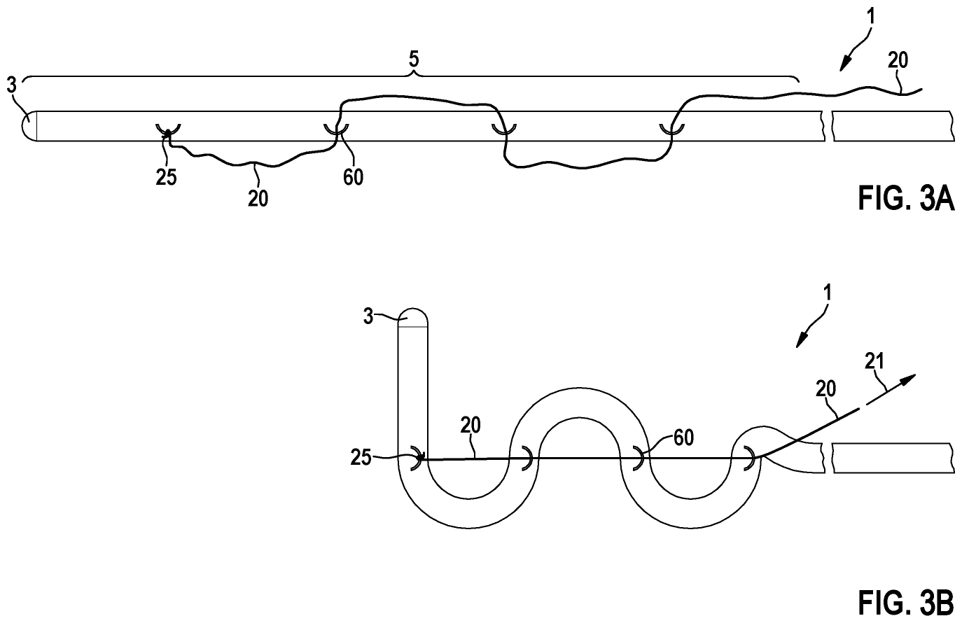

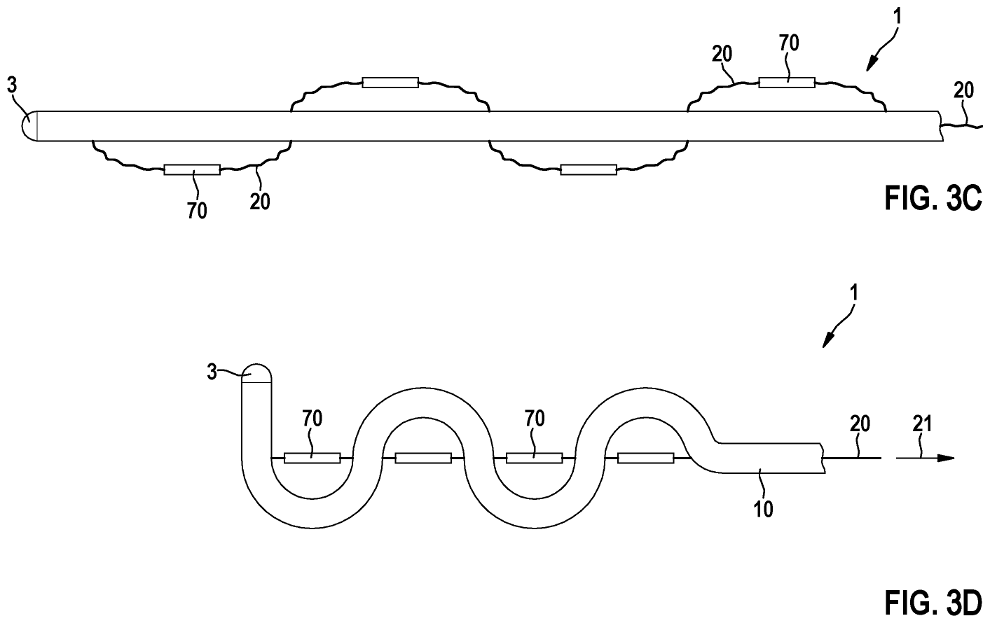

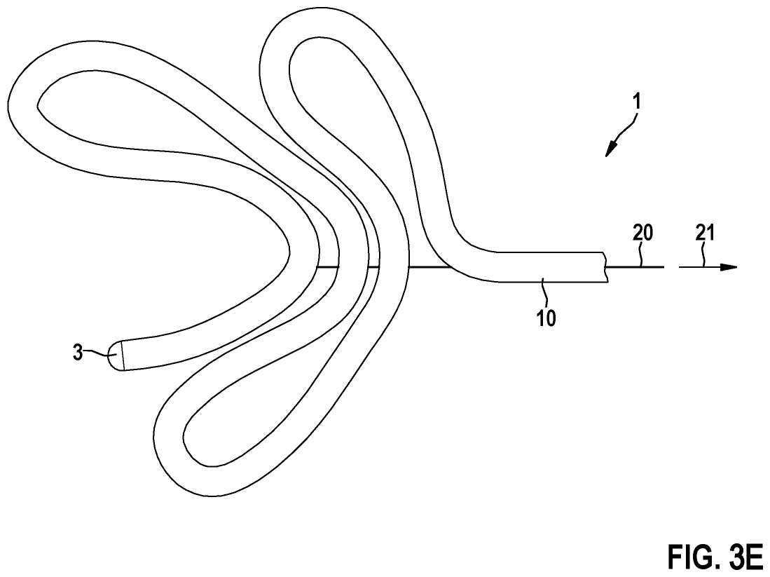

FIGS. 3A to 3E show electrode leads 1 by which a meander can be formed. For this purpose, the electrode lead 1 shown in FIG. 3A comprises multiple guide elements 60 arranged along the electrode lead body 10 thereof. The pulling element 20, which, in turn, is connected at the distal end 3 by the joining site 25 to the electrode lead 1, is inserted into the guide elements 60 so as to extend alternately on both sides of the electrode lead body 10. When a tensile force 21 is now exerted onto the pulling element 20, a meander-shaped region of the electrode lead 1 is formed, as shown in FIG. 3B. The meander-shaped portion of the electrode lead advantageously comprises a coil 30, serving as the electrode pole.

So as to stabilize the meanders shown in FIG. 3B, spacers 70 in the form of sleeves can additionally be threaded onto the pulling element 20 (see FIG. 3C). These sleeves 70 cause a specific, predefined distance to be maintained between the individual branches of the meander (see FIG. 3D) when a tensile force 21 is exerted onto the pulling element 20. If the spacers 70 are dispensed with, the individual branches of the meanders contract, as shown in FIG. 3E, when, proceeding from the situation shown in FIG. 3B, an additional tensile force 21 is exerted onto the pulling element.

Both final configurations (FIGS. 3D and 3E) are advantageous to use as counter poles for a subcutaneous defibrillator.

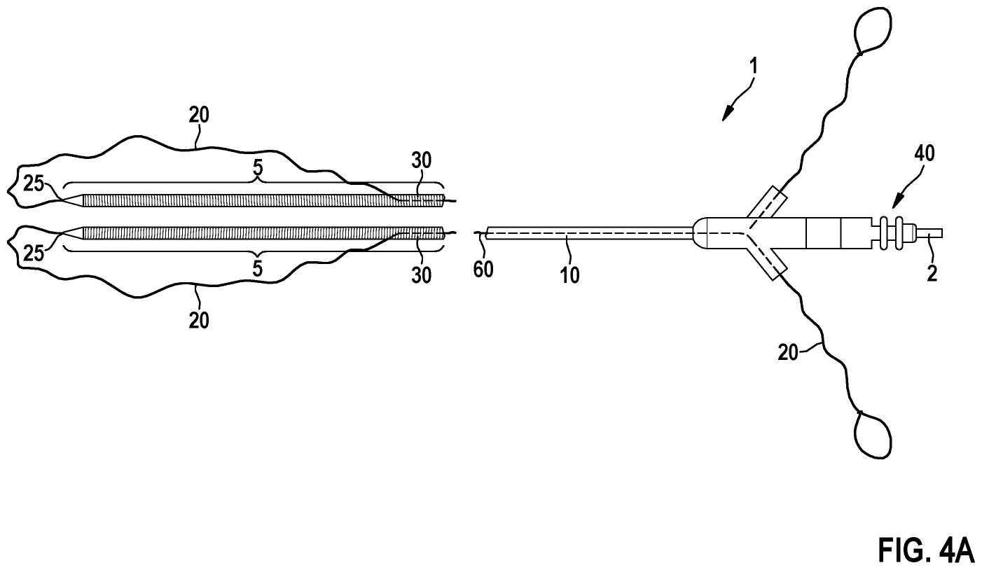

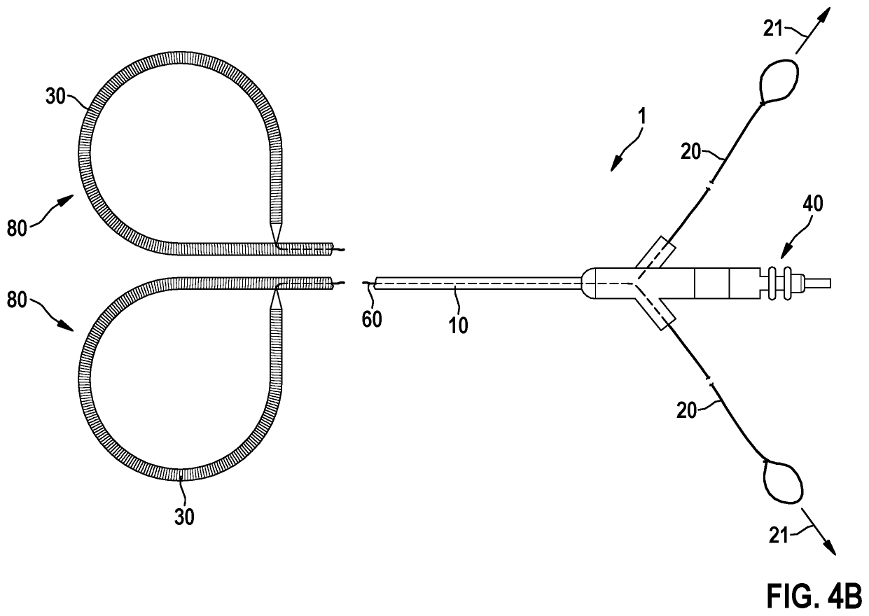

FIG. 4A shows an electrode lead 1 on which two electrode poles are arranged in the form of a coil 30 in the distal region 5. This principle is not limited to two parallel coiled electrode poles 30 in the distal region 5 of the electrode lead 1. The electrode lead 1 shown in FIG. 4A furthermore comprises two pulling elements 20, so that the two distal regions 5 (“fingers”) are curved individually—analogously to FIG. 1A/1B—to form a loop 80 when a tensile force 21 is exerted on the respective pulling element 20 (see FIG. 4B).

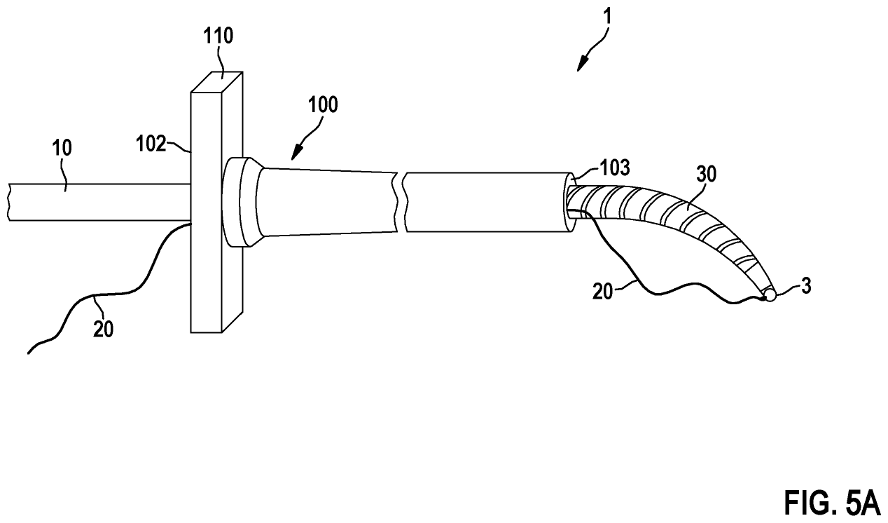

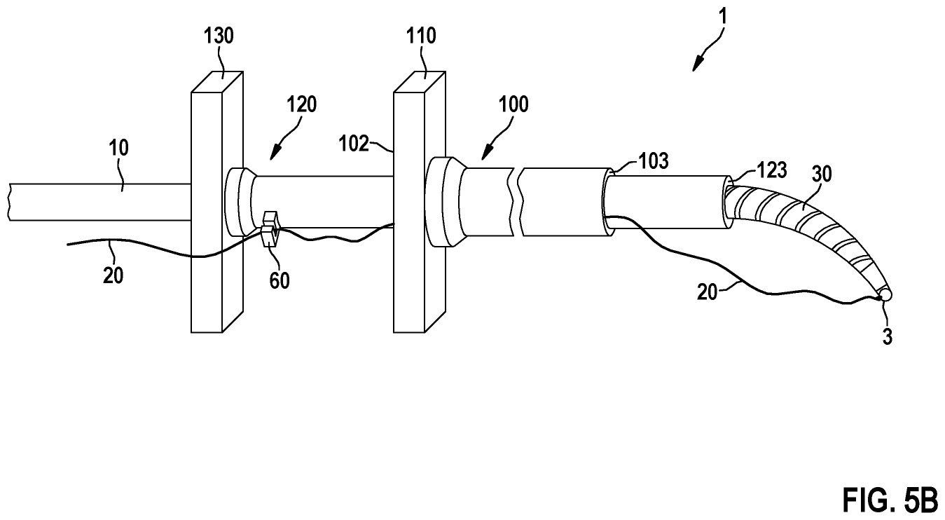

As is shown in FIG. 5A, as an alternative to the guide elements 60 arranged on the electrode lead body 10 of the electrode lead 1, it is also possible to use systems comprising an electrode lead 1 and an introducer sheath 100. In the system shown in FIG. 5A, the electrode lead 1 is inserted into an introducer sheath 100. At the distal end 103 of the introducer sheath 100, the pulling element 20 of the electrode lead 1 enters the inner lumen of the introducer sheath 100 simultaneously with the electrode lead 1. During the joint insertion of the sheath 100 together with the electrode lead 1 into the body, the electrode lead 1 can be pulled back into the sheath 100. So as to allow the introducer sheath 100 to move better, the sheath comprises a handle 110 at the proximal end 102 thereof. When the introducer sheath 100 is pulled back in relation to the electrode lead 1, as is shown in FIG. 5A, the distal section 5 of the electrode lead 1 likewise forms a loop 80, analogously to FIG. 1B, as soon as a tensile force 21 is exerted onto the pulling element 20.

FIG. 5B shows a system comprising an introducer sheath 100 and an electrode lead 1 by which the coiled electrode pole 30 can be brought into a U shape or an elongated loop 80. For this purpose, the introducer sheath 100 additionally comprises an inner catheter 120, which is arranged inside the lumen of the sheath 100. To provide better maneuverability of the two catheters 100 and 120 arranged inside one another, a handle 110 is provided on the introducer sheath 100, and a handle 130 is also provided on the inner catheter 120. As a result of the handles 110 and 130, it is possible to move the two catheters 100 and 120 relative to one another. The electrode lead 1, in turn, is arranged inside the lumen of the inner catheter 120. At the distal end 103 of the introducer sheath 100, the pulling element 20 of the electrode lead 1 enters the inner lumen of the introducer sheath 100 simultaneously with the inner catheter 120.

Proceeding from the configuration of the two catheters 100 and 120 with respect to one another shown in FIG. 5B, a U is created, and ultimately an elongated loop, when—as is shown in FIG. 1B—a tensile force 21 is exerted onto the pulling element 20. The length of the U or of the elongated loop 80 is dependent on the distance by which the catheters 100 and 120 were displaced relative to one another. The longer the distal end 123 of the inner catheter 120 which protrudes at the distal end 103 of the introducer sheath 100 is selected, the longer the U or the elongated loop will be.

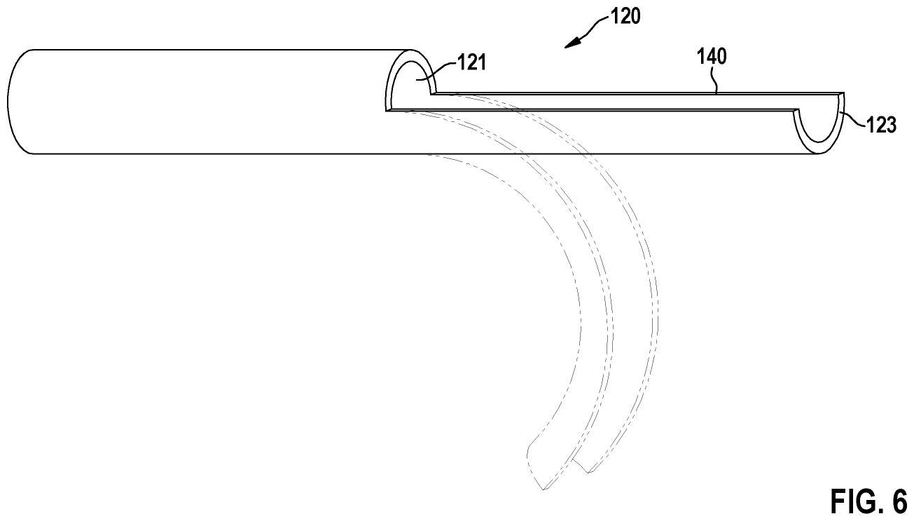

FIG. 6 shows the distal end 123 of the catheter 120 together with the lumen 121 extending on the inside thereof. Moreover, this catheter comprises a guiding aid 140. The guiding aid is designed in the form of a half shell or trough. It supports the formation of a clean curve, and consequently a clean formation of a loop 80 or a clean U shape. Furthermore, the guiding aid 140 helps to maintain the formation of the curve in the desired plane.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable extravascular electrode lead, comprising:
an electrode lead body extending from a distal end of the electrode lead to a proximal end of the electrode lead;
an elongated pulling element having a first end and a second end,
the pulling element, with the first end thereof, being connected to the electrode lead body by means of a joining site at the distal end of the electrode lead body of the electrode lead or in the distal region of the electrode lead,
the pulling element, in the elongated state of the electrode lead, extending from the joining site to the proximal end of the electrode lead and, at least in the distal region of the electrode lead, extending outside the electrode lead body, and
a tensile force exerted onto the second end of the pulling element exerting a bending moment onto the distal region of the electrode lead, which results in bending of the distal region of the electrode lead.

2. The implantable extravascular electrode lead according to claim 1, wherein a guide element, by which the pulling element is guided on the electrode lead body or in the electrode lead body, is arranged on the electrode lead body.

3. The implantable extravascular electrode lead according to claim 2, wherein the guide element is the designed in the form of a ring, an eyelet or a sleeve.

4. The implantable extravascular electrode lead according to claim 2, wherein the guide element is designed in the form of a channel inside the electrode lead body, which extends at least along a portion of the electrode lead body.

5. An implantable extravascular electrode lead according to claim 2, wherein the distance between the guide element and the joining site along the electrode lead body is between 30 mm and 800 mm.

6. An implantable extravascular electrode lead according to claim 1, wherein an electrode pole in the form of a shock coil is arranged on the electrode lead body between the joining site and the guide element.

7. An implantable extravascular electrode lead according to claim 1, wherein an engagement device is arranged on the electrode lead body close to the guide element or as part of the guide element, and a mating piece for the engagement device is arranged at the distal end of the electrode lead or at the joining site, the mating piece for the engagement device being designed to engage in the engagement device during insertion of the same so as to establish a mechanical connection between the mating piece for the engagement device and the engagement device.

8. The implantable extravascular electrode lead according to claim 7, wherein, in addition to a mechanical connection, also an electrical connection is established as a result of the connection between the engagement device and the mating piece for the engagement device.

9. An implantable extravascular electrode lead according to claim 2, wherein a plurality of guide elements are arranged spaced apart from one another on the electrode lead body in such a way that pulling on the pulling element causes the electrode lead body to contract in a meander-shaped manner.

10. An implantable extravascular electrode lead according to claim 1, wherein the pulling element is designed in the form of a thread, a cable, a wire, a bar or a rod.

11. An implantable extravascular electrode lead according to claim 1, wherein the pulling element is designed as a bar or a rod is precurved.

12. An implantable extravascular electrode lead according to claim 1, wherein the pulling element is resorbable.

13. An implantable extravascular electrode lead according to claim 1, wherein a plug is arranged on the electrode lead body at the proximal end of the electrode lead, and the plug is configured with a locking unit in such a way that the pulling element can be locked on the plug.

14. A system for inserting an implantable extravascular electrode lead, comprising an electrode lead according to claim 1 and a catheter having a lumen, wherein the electrode lead and the pulling element are arranged in the lumen of the catheter.
